(19) United States
(12) Patent Application Publication
(10) Pub. No.: US 2004/0059400 A1
(43) Pub. Date: Mar. 25, 2004

(54) Electric fever relief device

(76) Inventor: Ray-Ming Lin, Shinjuang City (TW)

Correspondence Address: Alan D. Kamrath, Rider, Bennett, Egan & Arundel, LLP, Suite 2000, 333 South Seventh Street, Minneapolis, MN, 55402, US

(21) Appl. No.: 10/254,243

(22) Filed: Sep. 25, 2002

(51) Int. Cl. A61F 7/00; A61F 7/12

(52) U.S. Cl. 607/109; 607/96

(57) ABSTRACT

A fever relief device includes body in which a thermoelectric cooler is received and the assembly of the body and the cooler is conveniently mounted to the head of the user who may adjust the direct current to control the temperature of the cooler so as to relieve the fever.

### FIELD OF THE INVENTION

The present invention relates to a fever relief device that uses a Peltier effect-thermoelectric cooler and the temperature can be adjusted by regulating direct current powering the cooler so as to relieve fever of patients.

### BACKGROUND OF THE INVENTION

A conventional way to relieve fever of a patient is to put a bag on the forehead of the patient and ice cubes or cool water is retained in the bag so as to absorb the heat from the patient to achieve the purpose of fever relief. There are water drops flowing from the forehead of the patient and is uncomfortable for the patient. The temperature of the bag can only last for a short period of time so that the bag has to be refill with ice cubes or water and this takes a lot of time and efforts. A fever relief pad was developed and can be conveniently adhered on the forehead of the patient. Nevertheless, this pad contacts the skin of the patent who could be sensitive to the pad or the adherent on the pad. Children peel the pads so often during using the pads and prolong the period of the fever.

The present invention intends to provide a fever relief device that is mounted to the head of a patient and the temperature can be adjusted and kept.

### SUMMARY OF THE INVENTION

In accordance with one aspect of the present invention, there is provided a fever relief device which comprises a body having a curve inside so as to contact a forehead of a patient and a Peltier effect-thermoelectric cooler is connected to the body. A cooling surface of the cooler is exposed to the inside of the body and in contact with the forehead of the patient. A heat transferring plate is connected to the cooler and an electric power supply received in the body is to power the cooler. A variable resistor is connected between the electric power supply and the cooler.

The primary object of the present invention is to provide a fever relief device that provides fever relief feature continuously.

Another object of the present invention is to provide a fever relief device that can be used repeatedly.

Yet another object of the present invention is to provide a fever relief device wherein the temperature can be adjusted.

A further object of the present invention is to provide a fever relief device that provides a comfortable fabric for the skin of the patient.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the fever relief device of the present invention;

FIG. 2 is an exploded view to show the fever relief device of the present invention;

FIG. 3 is a cross sectional view to show the fever relief device of the present invention;

FIG. 4 shows the fever relief device of the present invention is mounted to the head of a patient, and FIG. 5 shows the fever relief device of the present invention can be attached to an inside of a hat by hook-loop strips.

### DETAILED DESCRIPTION OF THE PREFERRED EMBODIMENT

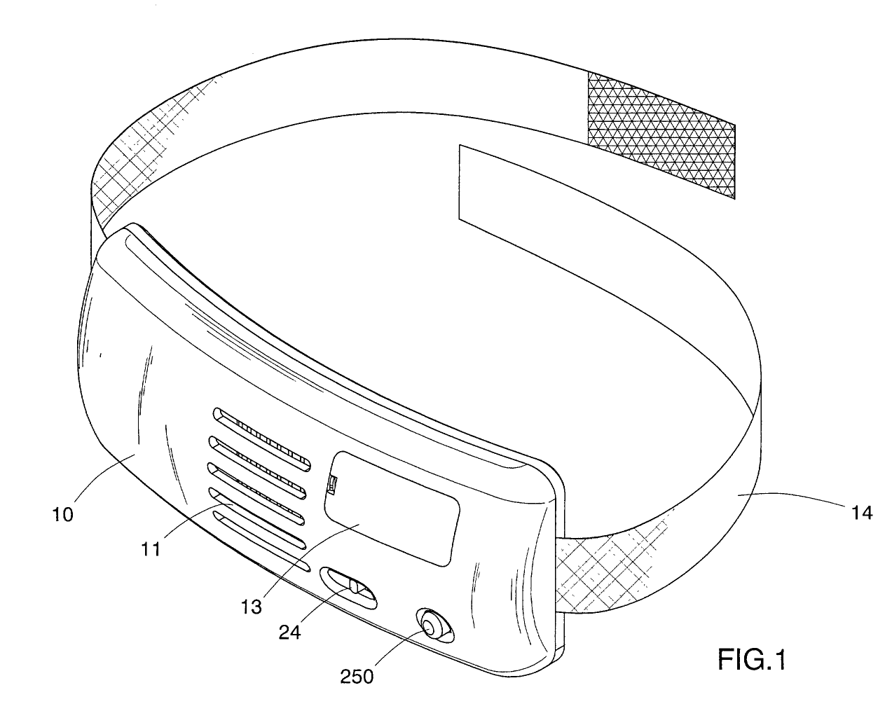

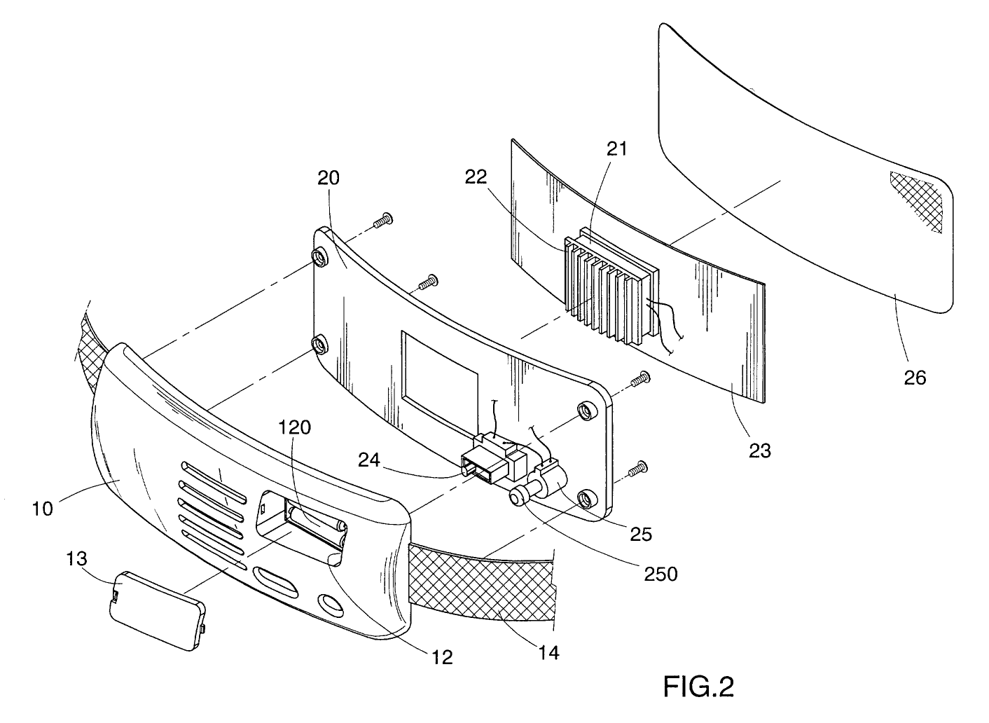

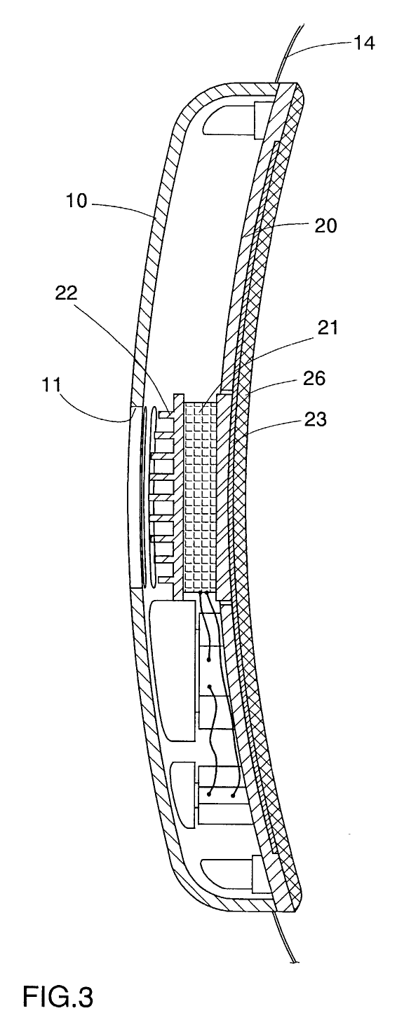

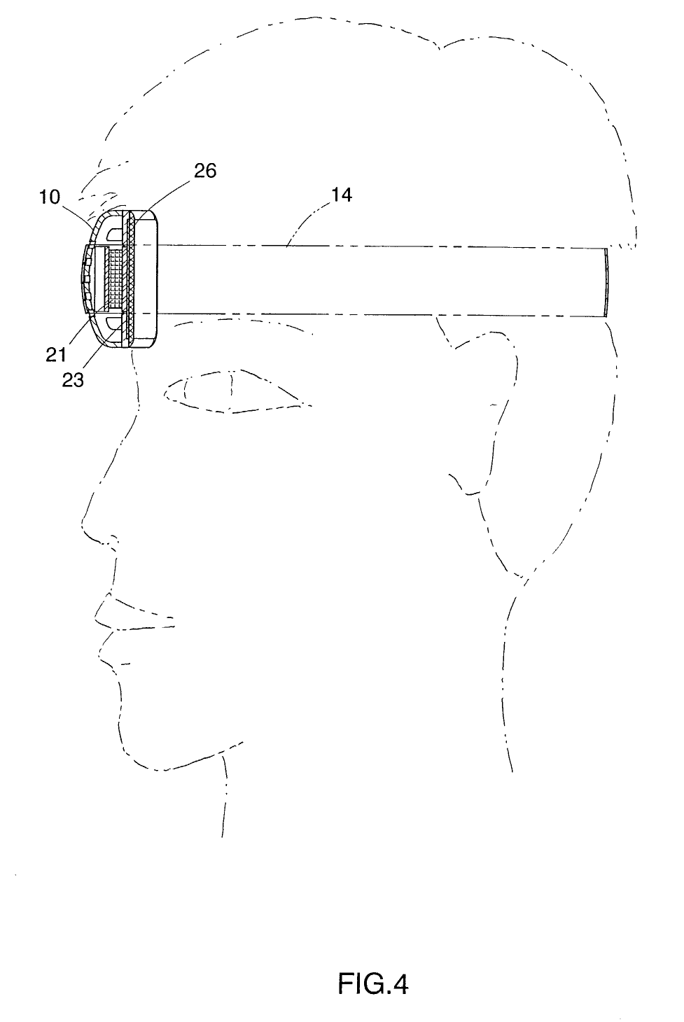

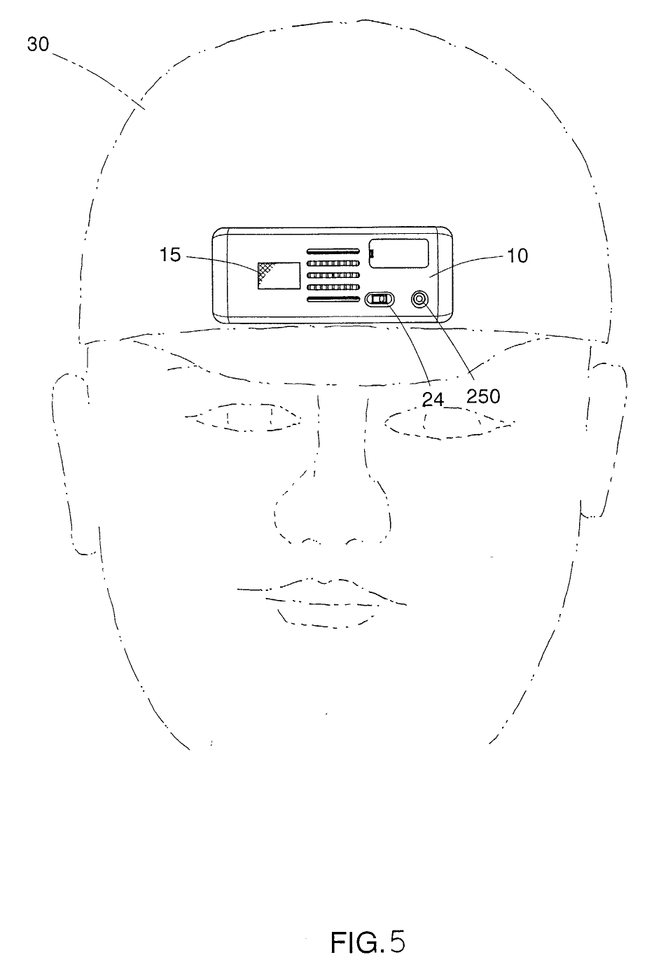

Referring to FIGS. 1 to 3, the fever relief device of the present invention comprises a hollow body 10 which has a curve inside so as to contact a forehead of a patient and two securing bands 14 are connected to two ends of the body 10. The body 10 is easily mounted to the head of the patient as shown in FIG. 4 by the securing bands 14. A plurality of ventilation slots 11 and a recess 12 are defined in an outside of the body 10. An electric power supply 120 which can be batteries are received in the recess 12 and a cap 13 is engaged with the recess 12.

A Peltier effect-thermoelectric cooler 21 is received in the body 10 and a cooling surface of the cooler 21 is exposed to the inside of the body 10. A metal heat transferring plate 23 is connected to the cooler 21 so that the cooling surface of the cooler 21 and the metal heat transferring plate 23 are in contact with the forehead of the patient. The electric power supply 120 in the body 10 powers the cooler 21 and a plurality of heat dispensing fins 22 are connected to the cooler 21 and located in opposite to the cooling surface of the cooler 21. The ventilation slots 11 are located in correspondence with the heat dispensing fins 22 so as to allow the heat generated from the cooler 21 to escape from the ventilation slots 11.

A board is located between the outside of the body 10 and the metal heat transferring plate 23. A variable resistor 25 and a switch 24 are connected to the board and located between the electric power supply 120 and the cooler 21. An adjusting switch 250 is connected to the variable resistor 25 so as to adjust the direct current to control the temperature of the cooler 21. The switch 24 and the adjusting switch 250 can be accessed via holes defined through the outside of the body 10. A fabric 26 made of soft material is connected to the heat transferring plate 23 such that the skin of the patient does not contact the cooling surface of the cooler 21 and the metal heat transferring plate 23 directly.

The cooler 21 can be controlled at a desired temperature by operating the adjusting switch 250 so that the cooler 21 is able to be operated continuously and the fabric 26 is friendly to the skin so that the device can be used by the patient who has sensitive skin.

Referring to FIG. 5, the fever relief device can be attached to an inside of a hat 30 by hook-loop strips 15 so as to be hidden by the hat 30 while in use.

While we have shown and described the embodiment in accordance with the present invention, it should be clear to those skilled in the art that further embodiments may be made without departing from the scope of the present invention.

What is claimed is:

1. A fever relief device comprising:
a body having a curve inside which is adapted to contact a forehead of a patient, a Peltier effect-thermoelectric cooler connected to the body and a cooling surface of the cooler exposed to the inside of the body, a heat transferring plate connected to the cooler, and
an electric power supply received in the body so as to power the cooler, a variable resistor connected between the electric power supply and the cooler.

2. The device as claimed in claim 1 further comprising a plurality of ventilation slots defined in an outside of the body.

3. The device as claimed in claim 1 further comprising two securing bands connected to two ends of the body.

4. The device as claimed in claim 1 further comprising a plurality of heat dispensing fins connected to the cooler and located in opposite to the cooling surface of the cooler.

5. The device as claimed in claim 1 further comprising an adjusting switch connected to the variable resistor.

6. The device as claimed in claim 1 further comprising a fabric connected to the heat transferring plate.

7. The device as claimed in claim 1 further comprising a recess defined in an outside of the body and a cap being engaged with the recess, the electric power supply received in the recess.

8. The device as claimed in claim 1, wherein the electric power supply is batteries.
